(19) United States
(12) Patent Application Publication
(10) Pub. No.: US 2024/0299378 A1
(43) Pub. Date: Sep. 12, 2024

(54) EMULSION FOR USE IN THE TREATMENT OF ROSACEA

(71) Applicant: EXINOV, Rambouillet (FR)

(72) Inventors: Frédéric LALLEMAND, Rambouillet (FR); Mathieu SCHMITT, Paris (FR)

(73) Assignee: EXINOV, Rambouillet (FR)

(21) Appl. No.: 18/547,587

(22) PCT Filed: Feb. 25, 2022
(86) PCT No.: PCT/EP2022/054758

§ 371 Date: Aug. 23, 2023

(30) Foreign Application Priority Data
Feb. 25, 2021 (EP) 21305221.0

(51) Int. Cl. A61K 31/498; A61K 9/06; A61K 31/20; A61K 31/365; A61K 31/4164; A61K 47/06; A61K 47/12; A61K 47/14; A61K 47/24; A61K 47/26; A61K 47/32; A61K 47/34; A61K 47/44; A61P 17/00

(52) U.S. Cl. CPC A61K 31/498; A61K 9/06; A61K 31/20; A61K 31/365; A61K 31/4164; A61K 47/06; A61K 47/12; A61K 47/14; A61K 47/24; A61K 47/26; A61K 47/32; A61K 47/34; A61K 47/44; A61P 17/00

(57) ABSTRACT

An emulsion including (i) at least one oil, (ii) at least one phospholipid and (iii) at least one polymeric surfactant. Typically, the oil is a mixture of triglycerides and fatty acids. Also, the use of the emulsion in the treatment and/or prevention of a skin disease, in particular rosacea. The emulsion is therapeutically active as such, even when it includes only excipients and no therapeutic agent against a skin disease. The emulsion may also be used as a topical vehicle for a therapeutic agent against a skin disease. Further, a method for the treatment and/or prevention of a skin disease in a subject in need thereof, the method including a step of administration of a therapeutically effective amount of the emulsion.

### FIELD OF INVENTION

The present invention relates to an emulsion useful in the treatment and/or prevention of a skin condition or skin disease, in particular rosacea.

### BACKGROUND OF INVENTION

Rosacea is a long-term skin condition that typically affects the face of the subject. Rosacea affects from 1 to 22% of a population, with higher prevalence in 30-50 years old, female and “Caucasians”. No cause from rosacea has been identified so far. Possible risk factors include a previous occurrence of the condition in the same family. The condition is believed to be worsen by external factors such as drinking of alcohol, exposure to high or low temperatures, physical exercise, menopause, psychological stress, consumption of spicy aliments, exposure to sunlight or topical use of steroid cream; or internal factors such as genetic predisposition, pre-existing skin condition, excess permeability of the skin or presence of Demodex mites or bacteria.

Diagnosis of rosacea is based on the detection of its symptoms. Symptoms of rosacea are skin redness, papules and/or pustules (often associated with chronic inflammation) and telangiectasia (widened venules—small blood vessels—cause threadlike red lines or patterns on the skin). The parts of the body that are typically affected by rosacea are cheeks, chin, forehead and the eye and nose. Rhinophyma is a severe form of rosacea wherein the nose becomes red and enlarged. Different strategies are considered for the management of rosacea, including a therapeutic approach and a cosmetic approach. The therapeutic approach of the symptoms involves the administration of a medicament containing a therapeutic agent (i.e., a drug substance) to the subject in need thereof, typically by oral route, in order to actively treat the symptoms of rosacea. No treatment exists that treats the cause of the disease. The cosmetic approach does not intend to treat the condition, but merely to hide the redness (“cover” the symptoms) and bring to the subject a sensation of comfort (as if the skin was healed, though it is not).

Rosacea is currently addressed through administration of a therapeutic agent acting on one of the physiopathological features of rosacea. The five major molecules indicated for the treatment of rosacea are ivermectin, metronidazole, brimonidine, azelaic acid and doxycycline. Ivermectin (Soolantra® medicine) is an anti-inflammatory and anti-parasitic agent from the avermectin family. Ivermectin is used in the topical treatment of rosacea for reducing the inflammation of the skin. The adverse effects of ivermectin include skin burning sensation, skin irritation, pruritus and skin dryness. Moreover, Soolantra® formulation contains substances with known adverse effects such as methyl para-hydroxybenzoate, propyl para-hydroxybenzoate and propylene glycols. Brimonidine (Mirvaso® medicine) is a highly selective alpha-2-adrenergic receptor agonist. Brimonidine is used in the topical treatment of rosacea for reducing erythema by means of direct cutaneous vasoconstriction. The adverse effects of brimonidine include erythema, pruritus, reddening and burning sensations of the skin; as well as less frequent but more severe adverse effects such as flush, pallor at the application site, hypotension or headache. Metronidazole is an antibiotic from the 5-nitro-imidazole family. Metronidazole is used in the topical treatment of rosacea for decreasing the population of Demodex mites in the skin. The adverse effects of metronidazole include skin dryness, erythema, pruritus, skin discomfort (e.g., burning, skin pain or stinging), skin irritation, worsening of rosacea; as well as less frequent but adverse effects such as hypaesthesia, paraesthesia or dysgeusia. Azelaic acid is an anti-inflammatory agent. Azelaic acid is used in the topical treatment of rosacea for reducing the inflammation of the skin. The adverse effects of azelaic acid include burning, stinging, skin dryness and numerous additional local side-effects. Doxycycline is an antibiotic from the tetracycline family. Doxycycline is used in the oral treatment of rosacea for decreasing the population of bacteria on the skin. The adverse effects of doxycycline include photosensitization reaction, skin rash, erythroderma, photo-onycholysis, hyperpigmentation, allergic reaction and allergic urticaria.

Thus, state-of-the-art medical solutions for the management of rosacea are responsible of significant adverse effects. Moreover, the therapeutic agents of the art act only on one physiopathological feature of rosacea, so that they have limited therapeutic efficacy and provide only limited relief to the patient. Hence, in spite of this skin condition being possibly first reported as early as 200 BC, there is a persistent need for improved compositions for use in the treatment and/or prevention of rosacea.

The Applicant surprisingly found out that an emulsion comprising a specific combination of three different excipients can be used in the treatment and/or prevention of a skin condition or skin disease such as rosacea, without the need of any of the therapeutic agents of the art. Therefore, the emulsion of the invention provides a novel and useful solution for the treatment of rosacea. The therapeutic effect of the emulsion of the invention is especially unexpected because each of the excipients as such (e.g., administered separately) would not provide any therapeutic benefit against rosacea, whereas the emulsion of the invention as a whole is therapeutically effective at alleviating the signs of rosacea.

### SUMMARY

The invention relates to an emulsion for use in the treatment and/or prevention of a skin disease, wherein the emulsion comprises (i) at least one oil, (ii) at least one phospholipid and (iii) at least one polymeric surfactant; wherein the emulsion is an oil-in-water emulsion; wherein the phospholipid does not comprise phosphatidylcholine (PC); and wherein the emulsion does not comprise vitamin D3, or a derivative or precursor thereof.

According to one embodiment, the oil is selected from triglycerides, mineral oils, fatty acids or mixtures thereof; preferably the oil comprises a mixture of triglycerides and fatty acids. According to one embodiment, the phospholipid is selected from dihexanoyl-phosphatidylglycerol, dioctanoyl-phosphatidylglycerol, didecanoyl-phosphatidylglycerol, dilauroyl-phosphatidylglycerol, lauroylmyristoyl-phosphatidylglycerol, dimyristoyl-phosphatidylglycerol, dipalmitoyl-phosphatidylglycerol (DPPG), myristoylpalmitoyl-phosphatidylglycerol, palmitoylstearoyl-phosphatidylglycerol, myristoylstearoyl-phosphatidylglycerol, distearoyl-phosphatidylglycerol, dioleoyl-phosphatidylglycerol, stearoyloleoyl-phosphatidylglycerol, palmitoyloleoyl-phosphatidylglycerol, dilinoleoyl-phosphatidylglycerol, dilinolenoyl-phosphatidylglycerol, diarachidonoyl-phosphatidylglycerol, didocosahexaenoyl-phosphatidylglycerol, a fatty acid ester of phosphatidylglycerol, and a mixture thereof; preferably the phospholipid is dipalmitoyl-phosphatidylglycerol (DPPG). According to one embodiment, the polymeric surfactant is selected from an alkyl aryl polyether alcohol, a block copolymer of ethylene oxide and propylene oxide, a polyvinyl alcohol, a polyoxyethylene fatty acid ester and a mixture thereof; preferably the polymeric surfactant is tyloxapol. In one embodiment, the emulsion comprises: at least oils being medium chain triglycerides (MCT), stearic acid, oleic acid, linoleic acid and alpha linolenic acid; at least one phospholipid being dipalmitoyl phosphatidylglycerol (DPPG); and at least one polymeric surfactant being tyloxapol.

According to one embodiment, the skin disease is rosacea, preferably papulopustular rosacea (subtype II rosacea). According to one embodiment, the treatment and/or prevention comprises a step of topical administration of the emulsion onto the skin of a subject.

The invention also relates to an emulsion comprising (i) at least one oil comprising a mixture of triglycerides and fatty acids, (ii) at least one phospholipid and (iii) at least one polymeric surfactant selected from an alkyl aryl polyether alcohol, a block copolymer of ethylene oxide and propylene oxide, a polyvinyl alcohol, a polyoxyethylene fatty acid ester and a mixture thereof; wherein the emulsion is an oil-in-water emulsion; wherein the phospholipid does not comprise phosphatidylcholine (PC); and wherein the emulsion does not comprise vitamin D3, or a derivative or precursor thereof.

According to one embodiment, the oil is a mixture of medium chain triglycerides (MCT), stearic acid, oleic acid, linoleic acid and alpha linolenic acid. According to one embodiment, the phospholipid is dipalmitoyl phosphatidylglycerol (DPPG). According to one embodiment, the polymeric surfactant is tyloxapol. In one embodiment, the emulsion comprises: at least oils being medium chain triglycerides (MCT), stearic acid, oleic acid, linoleic acid and alpha linolenic acid; at least one phospholipid being dipalmitoyl phosphatidylglycerol (DPPG); and at least one polymeric surfactant being tyloxapol. In one embodiment, the emulsion comprises: at least oils being medium chain triglycerides (MCT), linseed oil, corn oil, cetearyl alcohol, lanolin and squalane; at least one phospholipid being dipalmitoyl phosphatidylglycerol (DPPG); at least polymeric surfactants being tyloxapol and polyethylene glycol 20 stearate; at least one viscosifier being trehalose; at least one pH adjusting agent being ascorbic acid; and water. According to a first embodiment, the emulsion does not comprise any therapeutic agent against a skin disease. According to a second embodiment, the emulsion comprises another therapeutic agent against a skin disease; preferably a therapeutic agent selected from ivermectin, brimonidine, metronidazole, doxycycline, azelaic acid, tea tree oil and mixtures thereof.

### Definitions

In the present invention, the following terms have the following meanings:
“About” placed in front of a number denotes more or less 10% of the nominal value of this number.
“Administration” or a variant thereof (e.g., “administering”) refers to the provision of a composition to a subject in whom/which condition or disease, and/or its attendant symptoms, is to be treated and/or prevented.
“Alkyl” refers to a linear or branched hydrocarbyl radical of formula CₙH₂ₙ₊₁ wherein n is an integer greater than or equal to 1. Alkyl groups typically comprise from 1 to 16 carbon atoms, preferably from 1 to 12 carbon atoms, more preferably from 2 to 10 carbon atoms, more preferably from 3 to 8 carbon atoms, more preferably from 4 to 6 carbon atoms. Non-limitative examples of alkyl groups include methyl, ethyl, propyl, butyl, pentyl, hexyl, heptyl, octyl, nonyl, decyl, undecyl, dodecyl and isomers thereof
“Aryl” refers to a polyunsaturated, aromatic hydrocarbyl group having a single ring (such as phenyl) or multiple aromatic rings fused together (such as naphthyl) or linked covalently. Aryl groups typically comprise from 5 to 12 carbon atoms, preferably from 6 to 10 carbon atoms, more preferably 6 carbon atoms. Non-limiting examples of aryl groups include phenyl, benzyl, biphenyl, biphenylenyl and naphthalenyl.
“Comprise” or a variant thereof (e.g., “comprises”, “comprising”) is used herein according to common patent application drafting terminology. Hence, “comprise” preceded by an object and followed by a constituent means that the presence of a constituent in the object is required (typically as a component of a composition), but without excluding the presence of any further constituent(s) in the object. Moreover, any occurrence of “comprise” or a variant thereof herein also encompasses narrower expression “substantially consist”, further narrower expression “consist of” and any variants thereof (e.g., “consists”, “consisting”), unless otherwise stated.
“Emulsion” refers to a colloidal mixture of two liquid substances which under normal conditions are immiscible (such as water and oil), but which through specific operations succeed in having a macroscopically homogeneous, but microscopically heterogeneous aspect. One of the substances (dispersed phase) will be dispersed in the second substance (continuous phase) as droplets or liquid particles. A nanoemulsion is an emulsion that includes droplets or liquid particles ranging in size from 100 nm to 600 nm. A microemulsion comprises droplets ranging in size from 1 μm to 100 μm. When the dispersed phase is lipophilic (typically an oil) and the continuous phase is hydrophilic (typically an aqueous solution), the emulsion is an “oil-in-water emulsion”. When the dispersed phase is hydrophilic (typically an aqueous solution) and the continuous phase is lipophilic (typically an oil), the emulsion is an “water-in-oil emulsion”.
“Excipient” refers a substance present in a cosmetic or pharmaceutical composition, typically in a medicine. An excipient has no therapeutic effect on its own, which means that when administered to a patient alone (as such), i.e., without any other excipient or therapeutic agent, it does not treat nor prevent the condition or disease. In particular, an excipient administered alone to a patient with a skin condition is not therapeutically effective in the treatment and/or prevention of a skin condition or skin disease. Excipients are generally included in a composition for administration (e.g., as pharmaceutical vehicle), stabilization, bulking up or manufacturing purposes. Excipients are typically used in association with a therapeutic agent (API) for the treatment and/or prevention of a disease.
“Fatty acid” or “free fatty acid” are synonyms and refer to a carboxylic acid with a long aliphatic hydrocarbon chain. A fatty acid may be saturated or unsaturated, branched or unbranched. Fatty acids typically have an unbranched chain. Fatty acids typically have an even number of carbon atoms. Fatty acids typically have from 4 to 28 carbon atoms, preferably from 6 to 24 carbon atoms, more preferably from 8 to 20 carbon atoms.
“Fatty acid ester” refers to an ester of a fatty acid.
“Fatty alcohol” refers to an alcohol with a long aliphatic hydrocarbon chain. A fatty alcohol may be saturated or unsaturated, branched or unbranched. Fatty alcohols typically have an unbranched chain. Fatty alcohols typically have an even number of carbon atoms. Fatty alcohols typically have from 4 to 28 carbon atoms, preferably from 6 to 24 carbon atoms, more preferably from 8 to 20 carbon atoms.
“Human” refers to a subject of both genders and at any stage of development (i.e., neonate, infant, juvenile, adolescent and adult).
“Lecithin” refers to a mixture of glycerophospholipids found in animal and plant tissues. Glycerophospholipids components of lecithin typically include phosphatidylcholine (PC), phosphatidylethanolamine (PE), phosphatidylinositol (PI), phosphatidylserine (PS), and phosphatidic acid (PA).
“Patient” refers to a warm-blooded animal, preferably a mammal, more preferably a human, who/which is awaiting the receipt of, or is receiving medical care or is/will be the object of a medical procedure.
“Pharmaceutically acceptable” means that the ingredients of a composition are compatible with each other and not deleterious to the patient to whom/which the composition is administered.
“Pharmaceutical composition” refers to a composition substantially consisting of pharmaceutically acceptable ingredients, preferably consisting of pharmaceutically acceptable ingredients.
“Polymeric surfactant” refers to macromolecules containing both hydrophilic and hydrophobic moieties in their structure, which are thus able to act as surfactants. Non-limitative examples of polymeric surfactants include amphiphilic di-block copolymers (wherein the hydrophilic and hydrophobic parts are in different monomers) and polymers made of amphiphilic monomers (wherein the hydrophilic and hydrophobic moieties are in the same monomer). Compared to conventional (non-polymeric) surfactants, the macromolecular nature of polymeric surfactants allows much diversity in structures and better control of surfactant properties such as hydrophilic/hydrophobic balance.
The terms “prevent”, “preventing” and “prevention”, as used herein, refer to a method of delaying or precluding the onset of a condition or disease and/or its attendant symptoms, barring a patient from acquiring a condition or disease, or reducing the risk for a patient of acquiring a condition or disease.
“Skin condition”, “cutaneous condition” and “dermatological condition” are synonyms and refer to any medical condition that affects the integumentary system including skin, hair, nails and related muscle and glands. Typically, the affected part of the integumentary system is skin.
“Skin disease”, “cutaneous disease” and “dermatological disease” are synonyms and refer to a condition of the integumentary system, preferably a skin condition, associated with specific symptoms and signs.
“Subject” refers to a warm-blooded animal, preferably a mammal, more preferably a human. The subject may be a “patient” as defined herein.
“Therapeutic agent” refers to a molecule or a substance whose administration to a subject treat and/or prevent a condition or disease, and/or its attendant symptoms (in short, the molecule or substance is “therapeutically effective”). In particular, a “therapeutic agent against . . . ” or a variant thereof (e.g., “the therapeutic agent is effective against . . . ”) preceding the name of a specific condition or disease refers to a molecule or a substance which administration to a subject treats and/or prevents the specific condition or disease (as specified), and/or its attendant symptoms. Typically, a therapeutic agent is a therapeutic agent against a skin condition or skin disease, preferably against rosacea. Typically, a therapeutic agent is therapeutically effective when administered alone (as such) to the patient (i.e., no other therapeutic agent is present in the composition nor is separately administered to the patient). Typically, a therapeutic agent is therapeutically effective in a topical composition even when present in low amounts, for example less than 10% w/w, less than 1% w/w, less than 0.5% w/w, less than 0.1% w/w, less than 0.05% w/w or less than 0.01% w/w, the effective amount depending on the therapeutic agent and the treated condition or disease. A therapeutic agent may have a pharmacological activity based on an interaction with at least one biological receptor and/or biological target.
“Therapeutically effective amount” (or more simply an “effective amount”) refers to the amount of a composition and/or of a therapeutic agent that is sufficient to achieve the desired therapeutic and/or prophylactic effect in the patient to which/whom it is administered.
“Topical” refers to the delivery, administration or application of a composition directly to a skin or mucous membrane for a localized effect.
“Topical composition” refers to a composition that is suitable for topical administration.
“Triglyceride” refers to fatty acid triesters of glycerol.
The terms “treat”, “treating” and “treatment”, as used herein, are meant to include alleviating, attenuating and/or abrogating a condition or disease and/or its attendant symptoms. Treating thus includes controlling one of the many factors implicated in the disease development. Treating thus include alleviating, attenuating and/or abrogating at least one symptom of the disease without acting on the cause of the disease.
“w/w” following a percentage value, unless otherwise stated, that said percentage value is in weight by total weight of the emulsion.

### DETAILED DESCRIPTION

### Emulsion

The invention relates to an emulsion comprising (i) at least one oil, (ii) at least one phospholipid and (iii) at least one polymeric surfactant.

In the emulsion according to the invention, the oil, the phospholipid and the polymeric surfactant are “excipients”, therefore they have no therapeutic effect of their own when administered alone to a subject. However, the Applicant surprisingly found out that an emulsion comprising this specific combination of three different excipients provides potent treatment and/or prevention of rosacea.

Although some substances may rarely qualify simultaneously as an oil, a phospholipid and/or polymeric surfactant, in the invention the oil, the phospholipid and the polymeric surfactant refer necessarily to three different substances, i.e., the “oil” component cannot be the same time the “phospholipid” component or “polymeric surfactant” component, etc. In others words, the emulsion of the invention systematically comprises at least three different components (excipients), at least one of them performing the function of the oil, another of them performing the function of the phospholipid, and the last one performing the function of the polymeric surfactant.

According to one embodiment, the emulsion is an oil-in-water emulsion. According to one embodiment, the emulsion comprises the dispersed phase (typically the oily phase) in an amount ranging from 5% to 65% w/w, preferably ranging from 10% to 45% w/w, more preferably ranging from 20% to 30% w/w. According to one embodiment, the emulsion comprises the continuous phase (typically the aqueous phase) in an amount ranging from 35% to 95% w/w, preferably ranging from 55% to 90% w/w, more preferably ranging from 70% to 80% w/w.

According to one preferred embodiment, the oil is comprised in the dispersed phase of the emulsion.

According to one embodiment, the phospholipid is comprised in the continuous phase of the emulsion.

According to one embodiment, the oil comprises:
vegetable oils including but not limited to almond oil, babassu oil, blackcurrant seed oil, borage oil, canola oil, castor oil, coconut oil, cod liver oil, corn oil, cottonseed oil, evening primrose oil, grapeseed oil, linseed oil, mustard seed oil, oat oil, olive oil, palm kernel oil, palm oil, peanut oil, rapeseed oil, safflower oil, sesame oil, soybean oil, sunflower oil, walnut oil or wheat germ oil;
animal oils including but not limited to lanolin, fish oil or shark liver oil;
semi-synthetic or synthetic oils including but not limited to partially hydrogenated or hydrogenated vegetal oils (e.g., hydrogenated castor oil, hydrogenated coconut oil, hydrogenated cottonseed oil, hydrogenated palm oil, hydrogenated soybean oil or hydrogenated soybean oil), squalane, silicone oils, mineral oils or petrolatum;
glycerol esters including but not limited to short chain (C₄-C₆) fatty acids mono- di- and triesters of glycerol, medium chain (C₈-C₁₂) fatty acids mono-, di- and triesters of glycerol, long chain (C₁₄ and more, typically C₁₄-C₂₈) fatty acids mono-, di- and triesters of glycerol, or triacetin;
propylene glycol esters including but not limited to short chain (C₄-C₆) fatty acids mono- and diesters of propylene glycol, medium chain (C₈-C₁₂) fatty acids mono- and diesters of propylene glycol or long chain (C₁₄ and more, typically C₁₄-C₂₈) fatty acids mono- and diesters of propylene glycol;
fatty alcohols such as cetyl alcohol, stearyl alcohol, myristyl alcohol, oleyl alcohol or octyldodecanol;
fatty acids including but not limited to saturated fatty acids such as butyric acid, caproic acid, caprylic acid, capric acid, lauric acid, myristic acid, palmitic acid, stearic acid, arachidic acid, behenic acid and lignoceric acid; and unsaturated fatty acids such as myristoleic acid, palmitoleic acid, sapienic acid, oleic acid, elaidic acid, vaccenic acid, paulinic acid, linoleic acid, linoelaidic acid, alpha-linolenic acid, gamma-linolenic acid, arachidonic acid, eicosapentaenoic acid, erucic acid, cervonic acid, nervonic acid or docosahexaenoic acid;
fatty acid esters including but not limited to methyl palmitate, ethyl oleate, isopropyl myristate, isopropyl palmitate, isopropyl stearate or isopropyl isostearate; and/or vitamin E and derivatives thereof including but not limited to vitamin E acetate, tocopherol or tocopherol acetate.

According to one embodiment, the oil comprises triglycerides, mineral oils and/or fatty acids.

According to one embodiment, the oil comprises triglycerides. Non-limitative examples of triglycerides include short chain (C₄-C₆) triglycerides, medium chain (C₈-C₁₂) triglycerides and long chain (C₁₄ and more, typically C₁₄-C₂₈) triglycerides. In one embodiment, the emulsion comprises triglycerides in an amount ranging from 1% to 25% w/w, preferably ranging from 3% to 15% w/w, more preferably ranging from 6.5% to 8.5% w/w. In one embodiment, the oil comprises medium chain triglycerides (MCT) (for example [CAS Number 438544-49-1]). MCT may for example be obtained by extraction of palm kernel oil or coconut oil.

According to one embodiment, the oil comprises mineral oils. Non-limitative examples of mineral oils include light mineral oil and heavy mineral oil. In one embodiment, the emulsion comprises mineral oils in an amount ranging from 1% to 25% w/w, preferably ranging from 3% to 15% w/w, more preferably ranging from 6.5% to 8.5% w/w.

According to one embodiment, the oil comprises fatty acids (i.e., free fatty acids). Non-limitative examples of fatty acids include palmitic acid, stearic acid, oleic acid, linoleic acid and alpha-linolenic acid. In one embodiment, the fatty acids are selected from saturated fatty acids such as palmitic acid or stearic acid. In one embodiment, the fatty acids are selected from omega-9 fatty acids such as oleic acid. In one embodiment, the fatty acids are selected from omega-6 fatty acids such as gamma-linoleic acid. In one embodiment, the fatty acids are selected from omega-3 fatty acids such as alpha-linolenic acid. In one embodiment, the emulsion comprises fatty acids in an amount ranging from 1% to 15% w/w, preferably ranging from 2% to 10% w/w, more preferably ranging from 4% to 6% w/w.

According to one embodiment, the oil comprises vegetable oils. In one embodiment, the oil comprises linseed oil and/or corn oil. In one embodiment, the oil comprises linseed oil (for example [CAS Number 8001-26-1]), which is a source of free fatty acids. The fatty acids present in linseed oil are mainly palmitic acid (4-6% w/w), stearic acid (2-3% w/w), oleic acid (10-22% w/w), linoleic acid (12-18% w/w) and alpha-linolenic acid (56-71% w/w), in weight by weight of the total fatty acids. In one embodiment, the emulsion comprises linseed oil in an amount ranging from 0.5% to 10% w/w, preferably ranging from 1% to 6% w/w, more preferably ranging from 2% to 4% w/w. In one embodiment, the oil comprises corn oil (for example [CAS Number 8001-30-7]), which is a source of free fatty acids. The fatty acids present in corn oil are mainly palmitic acid (10-15% w/w), stearic acid (2-3% w/w), oleic acid (22-28% w/w) and linoleic acid (55-62% w/w), in weight by weight of the total fatty acids. In one embodiment, the emulsion comprises corn oil in an amount ranging from 0.5% to 10% w/w, preferably ranging from 1% to 6% w/w, more preferably ranging from 2% to 4% w/w.

According to one embodiment, the oil comprises cetearyl alcohol (for example [CAS Number 67762-27-0]). “Cetearyl alcohol” or “cetostearyl alcohol” is a mixture of fatty alcohols, mainly cetyl (C16) alcohol and stearyl (C18) alcohol. In one embodiment, the emulsion comprises, typically in the dispersed phase, an emulsifying wax NF (USP-NF), e.g., Polawax™ NF. “Emulsifying wax NF” is a mixture of cetearyl alcohol and a polyoxyethylene sorbitan fatty acid esters, typically polyethylene glycol-20 stearate (PEG-20 stearate). Emulsifying wax NF is a commonly used cosmetic emulsifying ingredient in the art, defined for example by USP32-NF27 standard. According to USP32-NF27 standard, emulsifying wax NF has a melting range of 50-54° C., a pH (3% dispersion) of 5.5-7.0, a hydroxyl value of 178-192, an iodine value lower or equal to 3.5 and a saponification value lower or equal to 14. In one embodiment, the emulsion comprises the emulsifying wax in an amount ranging from 1% to 15% w/w, preferably ranging from 2% to 10% w/w, more preferably ranging from 4% to 6% w/w.

According to one embodiment, the oil comprises lanolin (also known as “wool yolk”, “wool wax”, or “wool grease”) (for example [CAS Number 8006-54-0]). Lanolin is a wax secreted by the sebaceous glands of wool-bearing animals. Lanolin may for example be obtained from the wool of domestic sheep breeds. In one embodiment, lanolin is highly purified grade. In one embodiment, the emulsion comprises lanolin in an amount ranging from 1% to 15% w/w, preferably ranging from 2% to 10% w/w, more preferably ranging from 3% to 5% w/w.

In one embodiment, the oil comprises squalane (for example [CAS Number 111-01-3]). Squalane is a hydrocarbon manufactured by hydrogenation of squalene. Squalene may be obtained from the livers of sharks (traditionally) or from environment-friendly sources such as olive oil, rice or sugar cane. In one embodiment, the emulsion comprises squalane in an amount ranging from 0.2% to 5% w/w, preferably ranging from 0.5% to 3% w/w, more preferably ranging from 1% to 2% w/w.

In one embodiment, the oil comprises methyl palmitate (for example [CAS Number 112-39-0]), which is the fatty acid methyl ester of palmitic acid. In one embodiment, the emulsion comprises methyl palmitate in an amount ranging from 0.01% to 1% w/w, preferably ranging from 0.05% to 0.5% w/w, more preferably ranging from 0.1% to 0.3% w/w.

In one embodiment, the oil comprises medium chain triglycerides (MCT), palmitic acid, stearic acid, oleic acid, linoleic acid and/or alpha-linolenic acid; wherein, optionally, the fatty acids are in the form of linseed oil and/or corn oil as described hereinabove. In one embodiment, the oil comprises medium chain triglycerides (MCT), palmitic acid, stearic acid, oleic acid, linoleic acid, alpha-linolenic acid (optionally as linseed oil and/or corn oil) and/or cetearyl alcohol (optionally as emulsifying wax NF). In one embodiment, the oil comprises medium chain triglycerides (MCT), palmitic acid, stearic acid, oleic acid, linoleic acid, alpha-linolenic acid (optionally as linseed oil and/or corn oil), cetearyl alcohol (optionally as emulsifying wax NF) and/or lanolin. In one embodiment, the oil comprises medium chain triglycerides (MCT), palmitic acid, stearic acid, oleic acid, linoleic acid, alpha-linolenic acid (optionally as linseed oil and/or corn oil), cetearyl alcohol (optionally as emulsifying wax NF), lanolin and/or squalane. In one embodiment, the oil comprises medium chain triglycerides (MCT), palmitic acid, stearic acid, oleic acid, linoleic acid, alpha-linolenic acid (optionally as linseed oil and/or corn oil), cetearyl alcohol (optionally as emulsifying wax NF), lanolin, squalane and/or methyl palmitate.

In one embodiment, the emulsion comprises a mixture of 2, 3, 4, 5, 6, 7, 8, 9, 10, 11 or 12 different oils. In one embodiment, the emulsion comprises a mixture of 6 different oils. In one embodiment, the emulsion comprises a mixture of 7 different oils. In one embodiment, the emulsion comprises a mixture of 8 different oils. In one embodiment, the emulsion comprises a total amount of oils ranging from 5% to 65% w/w, preferably from 6% to 60% w/w, more preferably ranging from 10% to 45% w/w, furthermore preferably ranging from 12% to 40% w/w, furthermore preferably ranging from 20% to 30% w/w, furthermore preferably ranging from 23% to 26% w/w.

According to one embodiment, the oil is not a phospholipid. According to one embodiment, the oil is not a polymeric surfactant.

According to one embodiment, the phospholipid is selected from: diacylglycerides (glycerophospholipids) such as phosphatidic acid (phosphatidate) (PA), phosphatidylethanolamine (cephalin) (PE), phosphatidylcholine (lecithin) (PC), phosphatidylserine (PS), phosphatidylinositol (PI), phosphatidylinositol phosphate (PIP), phosphatidylinositol bisphosphate (PIP2) or phosphatidylinositol trisphosphate (PIP3); phosphosphingolipids (sphingolipids) such as ceramide phosphorylcholine (sphingomyelin) (SPH), ceramide phosphorylethanolamine (sphingomyelin) (Cer-PE) or ceramide phosphoryllipid; and mixtures thereof.

According to one embodiment, the phospholipid is selected from dihexanoyl-phosphatidylglycerol, dioctanoyl-phosphatidylglycerol, didecanoyl-phosphatidylglycerol, dilauroyl-phosphatidylglycerol, lauroylmyristoyl-phosphatidylglycerol, dimyristoyl-phosphatidylglycerol, dipalmitoyl-phosphatidylglycerol (DPPG), myristoylpalmitoyl-phosphatidylglycerol, palmitoylstearoyl-phosphatidylglycerol, distearoyl-phosphatidylglycerol, myristoylstearoyl-phosphatidylglycerol, dioleoyl-phosphatidylglycerol, stearoyloleoyl-phosphatidylglycerol, palmitoyloleoyl-phosphatidylglycerol, dilinoleoyl-phosphatidylglycerol, dilinolenoyl-phosphatidylglycerol, diarachidonoyl-phosphatidylglycerol, didocosahexaenoyl-phosphatidylglycerol, a fatty acid ester of phosphatidylglycerol, and a mixture thereof. In one embodiment, the phospholipid is dipalmitoyl-phosphatidylglycerol (DPPG) (for example [CAS Number 4537-77-3]).

According to one embodiment, the phospholipid is not an oil. According to one embodiment, the phospholipid is not a polymeric surfactant.

According to one embodiment, the emulsion comprises a total amount of phospholipids ranging from 0.05% to 5% w/w, preferably ranging from 0.1% to 2% w/w, more preferably ranging from 0.2% to 1% w/w.

According to one embodiment, the polymeric surfactant is non-ionic.

According to one embodiment, the polymeric surfactant is selected from alkyl aryl polyether alcohols (such as tyloxapol), block copolymers of ethylene oxide and propylene oxide (e.g., poloxamers such as poloxamer 188 or poloxamer 407), polysorbates (such as polysorbate 80), polyoxyethylene alkyl esters (such as PEG-40 stearate), polyoxyethylene ethers (such as PEG-4 lauryl ether), ethoxylated alcohols (ethoxylated oleyl alcohol), polyoxyethylene fatty acid esters (PEG-6 stearate), polyoxyethylene sorbitan fatty acid esters (e.g., polysorbates such as polysorbate 80), polyoxyethylene glycol castor oil (such as PGE-40 hydrogenated castor oil), alkyl polyglycol ethers (such as Macrogol isotridecyl ether), alkyl polyglycol esters (such as PEG 25 propylene glycol stearate), polyoxylglycerides (such as caprylocaproyl macrogolglycerides), alkylpolyglycosides, polyvinyl alcohol or vitamin E polyethylene glycol succinate (such as tocopherol polyethylene glycol succinate (TPGS)); and mixtures thereof.

According to one embodiment, the polymeric surfactant is selected from an alkyl aryl polyether alcohol, a block copolymer of ethylene oxide and propylene oxide, a polyvinyl alcohol, a polyoxyethylene fatty acid ester and a mixture thereof. According to one embodiment, the polymeric surfactant is an alkyl aryl polyether alcohol. In one embodiment, the polymeric surfactant is a (C₆-C₁₀) alkyl aryl polyether alcohol. In one embodiment, the polymeric surfactant is an alkyl (C₆-C₁₀) aryl polyether alcohol. In one embodiment, the polymeric surfactant is an alkyl phenyl polyether alcohol. In one embodiment, the polymeric surfactant is a (C₆-C₁₀) alkyl phenyl polyether alcohol. In one embodiment, the polymeric surfactant is tyloxapol (for example [CAS Number 25301-02-4]). According to one embodiment, the emulsion comprises tyloxapol in an amount ranging from 0.1% to 10% w/w, preferably ranging from 0.2% to 5% w/w, more preferably ranging from 0.5% to 2% w/w.

According to one embodiment, the polymeric surfactant is a block copolymer of ethylene oxide and propylene oxide. According to one embodiment, the polymeric surfactant is a polyvinyl alcohol. According to one embodiment, the polymeric surfactant is a polyoxyethylene fatty acid ester. In one embodiment, the polymeric surfactant is a polyethylene glycol (PEG) stearate such as PEG-2 stearate, PEG-6 stearate, PEG-8 stearate, PEG-12 stearate, PEG-20 stearate, PEG-32 stearate, PEG-40 stearate, PEG-50 stearate, PEG-100 stearate and/or PEG-150 stearate. In one embodiment, the polymeric surfactant is PEG-20 stearate or PEG-40 stearate. In one embodiment, the polymeric surfactant is polyethylene glycol-20 stearate (PEG-20 stearate) (for example [CAS Number 9004-99-3]).

According to one embodiment, the emulsion comprises at least one polymeric surfactant being an alkyl aryl polyether alcohol and/or at least one polymeric surfactant being a polyethylene glycol (PEG) stearate. In one embodiment, the emulsion comprises tyloxapol and/or polyethylene glycol-20 stearate (PEG-20 stearate).

According to one embodiment, the polymeric surfactant is not an oil. According to one embodiment, the polymeric surfactant is not a phospholipid.

According to one embodiment, the emulsion comprises at least one solvent selected from aqueous solvents, organic solvents and mixtures thereof. Typically, the solvent acts as the continuous phase of the emulsion. In one embodiment, the emulsion comprises at least one aqueous solvent selected from water (e.g., buffered water, drinking water or purified water), hydroalcoholic solution, Ringer's solution, saline, sugar solution and a mixture thereof. In one embodiment, the emulsion comprises water. In one embodiment, the water is purified water (for example [CAS Number 7732-18-5]). In one embodiment, the emulsion comprises at least one organic solvent selected from alcohols such as ethanol, isopropanol, glycerine (glycerol), 2-propanol, propylene glycol or 2-(2-ethoxyethoxy)ethanol. In one embodiment, the emulsion comprises the solvent, preferably water, in an amount ranging from 50% to 90% w/w, preferably ranging from 55% to 85% w/w, more preferably ranging from 60% to 80% w/w.

According to one embodiment, the emulsion comprises at least one additive, preferably a pharmaceutically acceptable additive. According to one embodiment, the additive is selected from antioxidants, binders, buffers and pH adjusters, chelating agents, colorants, diluents and fillers (including thickening agents), emollients, emulsifiers, glidants and anti-adherents, humectants, lubricants, plasticizers, preservatives (including antimicrobials), propellants, protective colloids, solvents (including cosolvents), surfactants (including cosurfactants), suspending agents, viscosifiers (i.e., viscosity modifying and/or modulator agents) and mixtures thereof. These and others pharmaceutically acceptable additives are disclosed in “Remington: Essentials of pharmaceutics” (Edited by Linda Felton, Pharmaceutical Press 2013, London), the content of which is hereby incorporated by reference.

In one embodiment, the emulsion comprises at least one antioxidant. In one embodiment, the emulsion comprises at least one buffer and/or pH adjusting agent. In one embodiment, the emulsion comprises at least one emollient. In one embodiment, the emulsion comprises at least one emulsifier. In one embodiment, the emulsion comprises at least one humectant. In one embodiment, the emulsion comprises at least one preservative agent. In one embodiment, the emulsion comprises at least one propellant. In one embodiment, the emulsion at least one protective colloid. In one embodiment, the emulsion at least one non-aqueous solvent. In one embodiment, the emulsion comprises at least one suspending agent. In one embodiment, the emulsion comprises at least one viscosifier.

In one embodiment, the emulsion comprises at least one antioxidant selected from alpha-tocopherol (vitamin E), butylated hydroxyanisole (BHA), butylated hydroxytoluene (BHT), sodium bisulphite, sodium metabisulfite and a mixture thereof.

In one embodiment, the emulsion comprises at least one buffer and/or pH-adjusting agent selected from acetic acid/acetate, ascorbic acid (vitamin C), boric acid/borate (borax), carbonate, citric acid, citric acid/citrate, gluconate, histidine, hydrochloric acid, lactate, potassium hydroxide, phosphoric acid/sodium phosphate or disodium phosphate or tri-sodium phosphate, sodium carbonate, sodium hydrogen carbonate, sodium hydroxide (NaOH), tris-base buffer, tromethamine and a mixture thereof. In one embodiment, the emulsion comprises a pH-adjusting agent being ascorbic acid and/or sodium hydroxide. In one embodiment, the emulsion comprises the buffer and/or pH-adjusting agent in an amount ranging from 0.005% to 0.5% w/w, preferably ranging from 0.01% to 0.2% w/w, more preferably ranging from 0.02% to 0.1% w/w. In one embodiment, the emulsion comprises ascorbic acid (vitamin C) (for example [CAS Number 50-81-7]). In one embodiment, the emulsion comprises ascorbic acid in an amount ranging from 0.005% to 0.5% w/w, preferably ranging from 0.01% to 0.2% w/w, more preferably ranging from 0.02% to 0.1% w/w. In one embodiment, the emulsion comprises sodium hydroxide (NaOH). In one embodiment, the emulsion is adjusted at a pH of about 5 by progressive addition (e.g., dropwise addition) of sodium hydroxide.

In one embodiment, the emulsion comprises at least one preservative agent selected from benzalkonium chloride (BAK), boric acid, butylparaben, ethanol, methylparaben, phenol, phenethyl alcohol, potassium sorbate, propylene glycol, propylparaben, sorbic acid and a mixture thereof. In one embodiment, the emulsion comprises potassium sorbate (for example [CAS Number 24634-61-5]). In one embodiment, the emulsion comprises the preservative in an amount ranging from 0.01% to 1% w/w, preferably ranging from 0.02% to 0.5% w/w, more preferably ranging from 0.05% to 0.2% w/w. In one embodiment, the emulsion does not comprise any preservative agent, i.e., the composition is “preservative free” or “self-preserved”.

In one embodiment, the emulsion comprises at least one viscosifier selected from acacia, agar, sodium alginate, bentonite, carbomer, carboxymethyl cellulose sodium (CMC), guar gum, xanthan gum, hydroxypropyl cellulose (HPC), hypromellose (HPMC), methylcellulose, pectin, glycerol, sucrose, trehalose, and a mixture thereof. In one embodiment, the emulsion comprises the viscosifier in an amount ranging from 0.5% to 15% w/w, preferably ranging from 1% to 10% w/w, more preferably ranging from 2.5% to 7.5% w/w. In one embodiment, the emulsion comprises trehalose (for example [CAS Number 6138-23-4]). In one embodiment, the emulsion comprises trehalose in an amount ranging from 0.5% to 15% w/w, preferably ranging from 1% to 10% w/w, more preferably ranging from 3% to 7% w/w. In one embodiment, the emulsion comprises carbomer (for example [CAS Number 9003-01-4]). In one embodiment, the emulsion comprises carbomer in an amount ranging from 0.05% to 2% w/w, preferably ranging from 0.1% to 1% w/w, more preferably ranging from 0.25% to 0.75% w/w.

According to one embodiment, the emulsion comprises: at least oils being medium chain triglycerides (MCT), stearic acid, oleic acid, linoleic acid and alpha linolenic acid; at least one phospholipid being dipalmitoyl-phosphatidylglycerol (DPPG); and at least one polymeric surfactant being tyloxapol. In one embodiment, the emulsion comprises: at least oils being medium chain triglycerides (MCT), stearic acid, oleic acid, linoleic acid, alpha linolenic acid and cetearyl alcohol; at least one phospholipid being dipalmitoyl-phosphatidylglycerol (DPPG); and at least polymeric surfactants being tyloxapol and polyethylene glycol 20-stearate. In one embodiment, the emulsion comprises: at least oils being medium chain triglycerides (MCT), linseed oil, corn oil, cetearyl alcohol, lanolin and squalane; at least one phospholipid being dipalmitoyl-phosphatidylglycerol (DPPG); at least polymeric surfactants being tyloxapol and polyethylene glycol 20-stearate; at least one viscosifier being trehalose; at least one pH-adjusting agent being ascorbic acid; and water.

According to a first embodiment, the emulsion does not comprise any therapeutic agent against a skin condition or skin disease. In one embodiment, the emulsion does not comprise any therapeutic agent against rosacea. In this embodiment, the therapeutic effect arises only from the emulsion according to the invention, especially from the specific combination of excipients in the emulsion. Hence, the presence of a therapeutic agent is optional when treating and/or preventing rosacea with the emulsion according to the invention. In one embodiment, the emulsion does not comprise any therapeutic agent.

According to a second embodiment, the emulsion further comprises at least one therapeutic agent. In one embodiment, the emulsion further comprises at least one therapeutic agent against a skin condition or skin disease. In this embodiment, the therapeutic effect of the emulsion according to the invention is supplemented and/or reinforced by the use of a therapeutic agent commonly used in the art for treating a skin condition or skin disease, especially rosacea. In this embodiment, the emulsion according to the invention may also be used a pharmaceutical vehicle.

In one embodiment, the emulsion further comprises a therapeutic agent against rosacea. In one embodiment, the therapeutic agent against rosacea is selected from ivermectin (for example from 0.01 to 0.15% w/w), brimonidine (for example from 0.001 to 0.05% w/w), metronidazole (for example from 0.01 to 0.1% w/w), doxycycline (for example from 0.1 to 1.50% w/w), azelaic acid (for example from 5 to 20% w/w), tea tree oil and a mixture thereof. In one embodiment, the emulsion further comprises a therapeutic agent against acne. In one embodiment, the therapeutic agent against acne is selected from tretinoin (for example from 0.025% or 0.05% w/w), isotretinoin (for example 0.05% w/w), adapalene (0.1% w/w), benzoyl peroxide and a mixture thereof. In one embodiment, the emulsion further comprises a therapeutic agent against eczema. In one embodiment, the therapeutic agent against eczema is selected from corticosteroids, calcineurin inhibitors (such as tacrolimus or pimecrolimus), phosphodiesterase 4 (crisaborole) and a mixture thereof. In one embodiment, the emulsion further comprises a therapeutic agent against psoriasis. In one embodiment, the therapeutic agent against psoriasis is selected from methotrexate, cyclosporine A, apremilast, a retinoid and a mixture thereof.

According to one embodiment, the emulsion is a pharmaceutical composition. According to one embodiment, the emulsion is in the form of a paste, a gel, an ointment, a cream, a lotion, a foam or an aerosolized mixture. In one embodiment, the emulsion is in a semi-solid form selected from a gel, an ointment and a cream. In one embodiment, the emulsion is the form of a cream. The emulsion is advantageously a non-greasy cream. In one embodiment, the emulsion is a topical composition.

The emulsion is advantageously stable, i.e., it can be stored overtime without destabilization. Advantageously, the emulsion can be stored for 6 months, preferably 12 months, more preferably 24 months (“shelf-life”). The emulsion is advantageously sterilisable by methods known in the art, in accordance with safety requirements in the dermatological field. Advantageously, the emulsion keeps its structure and/or properties when sterilised, for example by autoclave from 100 to 150° C. during 1 to 60 min.

According to one embodiment, the emulsion does not comprise any therapeutic agent against an eye condition or eye disease. In one embodiment, the emulsion does not comprise any therapeutic agent.

According to one embodiment, the phospholipid does not comprise phosphatidylcholine (PC). In one embodiment, the emulsion does not comprise phosphatidylcholine (PC). According to one embodiment, the polymeric surfactant or the emulsion does not comprise polysorbate 80. In one embodiment, the polymeric surfactant or the emulsion does not comprise a polysorbate. According to one embodiment, the phospholipid or the emulsion does not comprise soybean lecithin or egg yolk lecithin. In one embodiment, the phospholipid or the emulsion does not comprise lecithin. According to one embodiment, the phospholipid or the emulsion does not comprise phosphatidylcholine (PC). According to one embodiment, the phospholipid or the emulsion does not comprise phosphatidylethanolamine (PE). According to one embodiment, the phospholipid or the emulsion does not comprise phosphatidylinositol (PI). According to one embodiment, the phospholipid or the emulsion does not comprise phosphatidylserine (PS). According to one embodiment, the phospholipid or the emulsion does not comprise phosphatidic acid (PA). According to one embodiment, the emulsion does not comprise liposomes. According to one embodiment, the emulsion does not comprise a polyoxyethylene castor oil. According to one embodiment, the polymeric surfactant or the emulsion does not comprise a dicarboxylic acid. According to one embodiment, the emulsion does not comprise solid lipid nanoparticles (SLNs). According to one embodiment, the emulsion does not comprise 7-dehydrocholesterol (7-DHC). According to one embodiment, the emulsion does not comprise vitamin D3, a derivative of vitamin D3, or a precursor of vitamin D3. In one embodiment, the emulsion does not comprise vitamin D, a derivative of vitamin D, or a precursor of vitamin D. In this context, “vitamin D” refers to any form of vitamin D, such as, for example, vitamin D2 (or cholecalciferol) or vitamin D3 (or ergocalciferol). In this context, a “precursor of vitamin D” refers to a provitamin of vitamin D, such as, for example, 7-dehydrocholesterol (7-DHC), which is a provitamin of vitamin D3. According to one embodiment, the emulsion does not comprise hyaluronic acid (HA) or a derivative thereof. According to one embodiment, the oil, the polymeric surfactant or the emulsion does not comprise a polyethylene glycol (PEG) glyceryl stearate. According to one embodiment, the emulsion does not comprise azelaic acid or a derivatives or prodrug thereof. According to one embodiment, the emulsion does not comprise any pigment. According to one embodiment, the emulsion does not comprise parabens such as methylparaben or propylparaben. According to one embodiment, the emulsion is not a nanoemulsion.

### Medical Uses

This invention also relates to an emulsion according to the invention, as described hereinabove, for use as a medicament.

This invention also relates to an emulsion according to the invention, as described hereinabove, for use in the treatment and/or prevention of a skin condition, in particular a skin disease. According to one embodiment, the skin condition is a dermatological condition, in particular a dermatological disease, involving an inflammation process. In one embodiment, the skin condition or skin disease is selected from rosacea, acne, blepharitis, eczema, psoriasis, and chemotherapy-induced side effects to skin (such as, for example, rash, itching, pruritis, and the like). In one embodiment, the skin condition or skin disease is selected from rosacea, acne, psoriasis and eczema. In one embodiment, the skin condition or skin disease is rosacea. In one embodiment, the rosacea is selected from erythematotelangiectatic rosacea (subtype I rosacea), papulopustular rosacea (subtype IIrosacea), phymatous rosacea (subtype III rosacea) and ocular rosacea (subtype IV rosacea). In one embodiment, the rosacea is erythematotelangiectatic rosacea (subtype I rosacea). In one embodiment, the rosacea is papulopustular rosacea (subtype II rosacea). In one embodiment, the rosacea is phymatous rosacea (subtype III rosacea). In one embodiment, the rosacea is ocular rosacea (subtype IV rosacea). In one embodiment, the rosacea is a variant of rosacea selected from pyoderma faciale (rosacea fulminans), rosacea conglobata and phymatous rosacea. In one embodiment, the subject with rosacea presents a high count of demodex mites, typically at least 10 mites within a surface of 3 mm per 3 mm of skin.

In one embodiment, the emulsion is administered onto the outer and/or inner eyelid (upper and/or lower eyelid) for use in the treatment of a skin condition or skin disease of the eye such as ocular rosacea or blepharitis. According to one embodiment, the emulsion is administered on a regimen of once per week to 9 times per day; preferably of twice per week to 6 times per day; more preferably of 3 times per week to 6 times per day. In one embodiment, the emulsion is administered on a regimen of once per two days to 3 times per day, preferably of once or twice per day. According to one embodiment, the emulsion is administered for at least 6 weeks. In one embodiment, the emulsion is administered for at least 3 months. In one embodiment, the emulsion is administered for at least 6 months.

It will be understood that the specific dose level and frequency of dosage for any particular patient may be varied and will depend upon a variety of factors including the activity of the emulsion, the metabolic stability and length of action of the emulsion, the age, body weight, general health, sex, diet, mode and time of administration, rate of excretion, drug combination, the severity of the particular condition, e.g., the particular skin disease, and the subject undergoing therapy.

The administration of the emulsion advantageously leads to healthier environment on and/or near the skin affected by the skin condition or skin disease. The administration of the emulsion advantageously leads to improvement in at least one symptom of the skin condition or skin disease. The administration of the emulsion advantageously leads to reduction of the inflammatory status of the skin condition or skin disease. Without wishing to be bound by any theory, the Applicant believes that the unexpected therapeutic activity of the emulsion according to the invention may derive at least in part from a mechanical effect thereof, i.e., by improving the mechanical properties of the skin affected by a skin condition or skin disease such as rosacea. Especially, the Applicant believes that improvement of mechanical properties may lead to healthier skin environment, reduction of symptoms and/or decrease of inflammation.

The emulsion advantageously does not form a long-lasting film when applied on the skin surface. Advantageously, the topical administration of the emulsion is comfortable for the subject.

This invention also relates to a method for the treatment and/or prevention of a skin condition, in particular a skin disease, in a subject in need thereof, the method comprising a step of administrating to said subject a therapeutically effective amount of an emulsion according to the invention, as described hereinabove. This invention also relates to the use of an emulsion according to the invention, as described hereinabove, in the manufacture of a medicament for the treatment and/or prevention of a skin condition, in particular a skin disease. This invention also relates to the use of an emulsion according to the invention, as described hereinabove, in the treatment and/or prevention of a skin condition, in particular a skin disease.

According to a first embodiment, the subject is not administered any therapeutic agent against a skin condition or skin disease. In one embodiment, the subject is not administered any therapeutic agent against rosacea. According to a second embodiment, the subject is further administered at least one therapeutic agent against a skin condition or skin disease. In one embodiment, the subject is further administered a therapeutic agent against rosacea. In one embodiment, the therapeutic agent is comprised in the emulsion according to the invention. In one embodiment, the emulsion and the therapeutic agent are administrated by means of sequential, simultaneous or separate administration to the subject, preferably as a combined preparation.

### Packaging and Devices

This invention also relates to a packaging comprising the emulsion according to the invention, as described hereinabove. In one embodiment, the packaging is a metered dose pump or a tube. This invention also relates to a dressing comprising the emulsion according to the invention, as described hereinabove. This invention also relates to a device comprising the emulsion according to the invention, as described hereinabove. In one embodiment, the device is a medical device.

### Manufacture

This invention also relates to a process for manufacturing an emulsion according to the invention comprising the following steps: (a) stirring together the components of the dispersed (oil) phase; (b) stirring together the components of the continuous (aqueous) phase; (c) adding the aqueous phase in the oily phase or the oily phase in the aqueous phase, thereby obtaining a pre-emulsion; and (d) decreasing the droplet size, thereby obtaining the final emulsion.

According to one embodiment, the stirring in stirring step (a) and/or (b) is magnetic stirring or stirring by means of a propeller. According to one embodiment, both phases are heated at about the same temperature before the addition step (c), for example between 40 and 90° C. According to one embodiment, the pre-emulsion is heated after the addition step (c), for example between 50 to 100° C. According to one embodiment, the droplet size is decreased in stirring step (d) by high shear mixing. According to one embodiment, the process further comprises a step (e) of homogenizing the emulsion between step (d) of decreasing the droplet size and obtaining the final emulsion. In one embodiment, the homogenizing step (e) is carried out by means of a microfluidizer. According to one embodiment, the pre-emulsion and/or the emulsion is cooled down before and/or after decreasing step (d) and/or before and/or after homogenizing step (e).

### BRIEF DESCRIPTION OF THE DRAWINGS

FIG. 1 is a histogram showing the effect on erythema of the administration of an emulsion Em-A according to the invention onto one side of the face of patients with rosacea, compared with the other side of the face where no emulsion is administered.

FIG. 2 is a histogram showing the effect on erythema of the administration of an emulsion Em-A according to the invention onto one side of the face of patients with rosacea presenting a high count of Demodex mites (specific subpopulation), compared with the other side of the face where no emulsion is administered.

FIG. 3 is a histogram showing the effect on erythema of the administration of an emulsion Em-A according to the invention onto one side of the face of patients with rosacea subtype II (specific subpopulation), compared with the other side of the face where no emulsion is administered.

FIG. 4 is a histogram showing the effect on Transepidermal Water Loss (TEWL) of the administration of an emulsion Em-A according to the invention onto one side of the face of patients with rosacea, compared with the other side of the face where no emulsion is administered. TEWL is a marker of the level of barrier properties of the skin (i.e., skin integrity).

FIG. 5 is a histogram showing the effect on the number of Demodex mites of the administration of an emulsion Em-A according to the invention onto one side of the face of patients with rosacea, compared with the other side of the face where no emulsion is administered.

### EXAMPLES

The present invention is further illustrated by the following examples.

### Example 1: Emulsion According to the Invention

### Materials and Methods

Materials: The components were purchased from commercial providers and used without further purification.

Methods—Manufacture of the emulsions: (1) Oil phase preparation: (1.1.) Lanolin and methyl palmitate were heated in a glass beaker at 42-48° C. (1.2.) The following oil phase components were weighed: lanolin, methyl palmitate, squalane, medium chain triglycerides (MCT), linseed oil, corn oil and emulsifying wax NF. (1.3.) The oil phase components were mixed and heated at 65-71° C. in a glass reactor. (2.) Aqueous phase preparation: (2.1.) The following aqueous phase components were weighed: purified water, dipalmitoyl-phosphatidylglycerol (DPPG), tyloxapol, potassium sorbate, ascorbic acid, trehalose and carbomer. (2.2.) The aqueous phase components (except ascorbic acid and carbomer) were mixed and heated at 65-71° C. (2.3.) Carbomer was added into the aqueous phase at 65-71° C. (3.) Emulsion manufacture: (3.1.) The oil phase was added into the aqueous phase and homogenised at 65-71° C. (3.2.) The homogenised mixture was stirred for 20 minutes using a propeller at 700 RPM at 65-71° C. (3.3.) The mixture was cooled down to 17-23° C. while stirring at 220 RPM. (3.4.) Ascorbic acid was added while stirring at 220 RPM. (3.5.) pH was adjusted using NaOH (1N) and homogenised to reach pH 4.6-5.4. (3.6.) The emulsion was hold inside the reactor until filling into the containers. (4.) Filling and labelling: (4.1.) Primary containers (bottles) were filled using appropriate equipment. (4.2.) The containers were labelled.

### Results

“Emulsion A” (“Em-A”) according to the invention has been prepared as described hereinabove, having the composition as indicated on Table 1.

TABLE 1
Emulsion A

| | | Em-A |
|---|---|---|
| Emulsion | Components | (% w/w) |
| Oil phase | Medium chain triglycerides (MCT) | 7.5 |
| | Emulsifying wax NF* | 5 |
| | Lanolin (highly purified grade) | 3.8 |
| | Linseed oil | 3.0 |
| | Corn oil | 2.5 |
| | Squalane | 1.5 |
| | Methyl palmitate | 0.2 |
| Aqueous | Trehalose | 5 |
| phase | Tyloxapol | 1 |
| | Carbomer | 0.5 |
| | Dipalmitoyl-phosphatidylglycerol | 0.5 |
| | (DPPG) | |
| | Potassium sorbate | 0.1 |
| | Ascorbic acid | 0.05 |
| | Purified water | q.s. 100 |
| | NaOH (1N) | q.s. pH 5 |

*The emulsifying wax NF is a mixture of cetearyl alcohol and PEG20-stearate.

Em-A does not comprise any therapeutic agent against rosacea and is however surprisingly effective in the treatment and/or prevention of rosacea as evidenced hereinafter.

Emulsions B, C, D and E according to the invention have been prepared as described hereinabove for Em-A, with further addition of a therapeutic agent against rosacea as indicated on Table 2.

TABLE 2
Emulsions B, C, D and E

| | Em-B | Em-C | Em-D | Em-E |
|---|---|---|---|---|
| Therapeutic agent | (% w/w) | (% w/w) | (% w/w) | (% w/w) |
| Ivermectin | 0.5 | — | — | — |
| Brimonidine | — | 0.05 | — | — |
| Metronidazole | — | — | 0.1 | — |
| Acid azelaic | — | — | — | 14.0 |

In Em-B, Em-C, Em-D, and Em-E, Em-A is used as a dermatological vehicle for topical administration of the therapeutic agent comprised therein. Em-A, Em-B, Em-C, Em-D, and Em-E are in semi-solid form (cream), which is convenient for topical administration on the skin.

### Example 2: In Vitro Cytotoxicity and Irritation of the Emulsion of the Invention

### Materials and Methods

Materials: Emulsion Em-A manufactured as described in Example 1 herein.

Methods: Cytotoxicity and skin irritation potential of the emulsions were both evaluated by means of a skin irritation assay using in vitro Episkin™ reconstructed human epidermis. The design of this assay was based on the “DB-ALM protocol No. 131. Episkin™ Skin irritation test method 15 min-42 hours”, and the standard operating procedure “Episkin™ Skin irritation test 42 hours Determination of IL-la concentration in the culture medium, version 1.2, published by ECVAM”. The Episkin™ model is a three-dimensional human skin model comprising a reconstructed epidermis with a functional stratum corneum. This assay is based on the fact that irritant chemicals are cytotoxic to the Episkin™ reconstructed human epidermis model after a short-term exposure. Irritant chemicals are able to penetrate the stratum corneum and are sufficiently cytotoxic to cause cell death in the underlying cell layers. Skin irritation test was carried out by topical application of tested emulsions on the surface of the epidermis for 15 minutes, followed by assessment of their effects on cell viability after a 42-hour recovery period. Cell viability was determined through cellular mitochondrial succinate dehydrogenase activity, measured (within the mitochondria of viable cells) by the reduction and conversion of a yellow dye, MTT [3-(4,5-dimethylthiazol-2-yl)-2,5-diphenyltetrazolium bromide], into a blue formazan salt. The formazan precipitate was extracted using acidic isopropanol and quantified by spectrophotometry. For each treated tissue, the viability was expressed as a percentage relative to the mean viability of the negative control tissues. Highly coloured chemicals and/or MTT reducers may interfere with cell viability measurements. In such cases, adapted controls for correction may be used. In addition, the concentration of the inflammatory mediator interleukin 1 alpha (IL-1α) was quantified from the culture medium retained following the 42 hours recovery period. This quantification, based on an ELISA assay, was performed for tested emulsions which were found with a mean relative viability higher than 50% following the MTT reduction assay, in order to confirm a non-irritant result or to override the non-irritant result. The upper limit for this test was 60 pg/mL.

### Results

Preliminary tests: In the preliminary tests, emulsion Em-A was confirmed as having no direct MTT reducing properties or colouring potential, hence the main test was technically feasible.

Main test: All acceptance criteria for the negative and positive controls were fulfilled. The study was therefore considered to be valid. Following a 15 minutes exposure to emulsion Em-A and 42 hours of recovery period, the relative mean viability of the tissues treated with the test item was 104% with a standard deviation of 2%. As the mean viability was higher than 50% after the MTT reduction, the results met the criteria for a non-irritant response. Moreover, as the mean viability was higher than 50% after the MTT reduction, the IL-1α concentrations in culture media samples retained from the three negative controls and the three Em-A-treated tissues (samples) were analysed by ELISA. The IL-1α concentration values of two samples were found below the limit of quantification (lower than 5.00 pg/mL). The IL-1α concentration in the remaining sample was 9.17 pg/mL, which remains significantly lower than 60 pg/mL (test threshold).

Therefore, these results clearly evidence that emulsion Em-A is non-cytotoxic and non-irritant to skin. Moreover, the unexpectedly low IL-1α levels strongly suggests that emulsion Em-A is beneficial for the management of inflammatory processes. This is especially relevant because patients treated for skin conditions or skin diseases such as rosacea often have a very sensitive skin. Thus, this test confirms the excellent tolerance and safety of the emulsion according to the invention for topical use and its suitability for multiple topical administrations on the skin.

### Example 3: In Vivo Sensitization of the Emulsion of the Invention

### Materials and Methods

Materials: Emulsion Em-A manufactured as described in Example 1 herein.

Methods: The emulsions were evaluated by means of mouse Local Lymph Node Assay (LLNA), which is the gold standard for the evaluation of the sensitization and sensibilization potential of compositions. The functional immune system of a mouse gives very robust data that have been proven to be predictable and transposable to human patients. Preliminary tests: Based on the results of the Preliminary Compatibility Test, the tested emulsions characteristics (e.g., when the emulsion is a dense cream), its use and following the Organisation for Economic Co-operation and Development (OECD) Guidelines, the best vehicle for the tested emulsions was considered as AOO (acetone:olive oil 4:1 (v/v) mixture). The tested emulsions at 100% (w/v) were suitable for the test using AOO as vehicle. Hence, the Preliminary Irritation/Toxicity Test was performed in CBA/CaOlaHsd mice using one dose (2 animals/dose) 100% (w/v) in AOO (acetone:olive oil 4:1 (v/v) mixture). Based on the observations recorded in this test, the 100% (w/v) in AOO dose was selected as top dose for the main test. Main test: In the main assay, twelve female CBA/CaOlaHsd mice were allocated to three groups of four animals each: (i) one group received the tested emulsions (formulated in AOO) at 100% (w/v) concentration; (ii) the negative control group received the vehicle (AOO) only; and (iii) the positive control group received 25% (w/v) HCA (dissolved in AOO). Tested emulsions were applied on the dorsal surface of ears of experimental animals (25 μL/ear using positive displacement pipette) for three consecutive days (days 1, 2 and 3). There was no treatment on days 4, 5 and 6. The cell proliferation in the local lymph nodes was assessed by measuring the incorporation of tritiated methyl thymidine (3HTdR) and the values obtained were used to calculate stimulation indices (SI) in comparison with the control group.

### Results

Neither mortality nor systemic toxicity was observed during the main study. No residual of emulsion Em-A was observed on the ears of the experimental animals. There was no indication of any irritation at the site of application. No unexpected effects of emulsion Em-A were observed on the mean body weights. The stimulation index (SI) value was 1.1 for Em-A at concentration of 100% (w/v). The result of the positive control substance (α-hexylcinnamaldehyde, HCA) dissolved in the same vehicle confirmed the appropriate performance of the assay. A lymphoproliferative response (SI=12.7) consistent with historical control data was noted for the positive control chemical, this result confirming the validity of the assay. Consequently, the stimulation index of emulsion Em-A (SI=1.1) was 10-fold lower than the threshold value (SI=12.7) defining a composition presenting no sensitization potential.

Therefore, these results clearly evidence that emulsion Em-A has no sensitization or sensibilization potential, and is consequently very safe. Emulsion Em-A is also devoid of any allergic potential. This is especially relevant considering that patients treated for skin conditions or skin diseases such as rosacea are prone to allergic reactions. Thus, this test confirms the excellent tolerance and safety of the emulsion according to the invention for topical use and its suitability for multiple topical administrations on the skin.

### Example 4: Clinical Evaluation of the Emulsion of the Invention

### Materials and Methods

Materials: Emulsion Em-A manufactured as described in Example 1 herein and stored in airless metered dosed bottles.

Methods: An exploratory clinical study was performed on subjects suffering from rosacea. Study design: A single centre, randomized, 6-week intra-individual (left vs right) comparison study was performed in adult healthy subjects with facial rosacea.

Emulsion Em-A was topically administered on half of the face of the patient twice per day for 6 weeks. Evaluation timepoints were baseline, week 2, week 4 and week 6. Study population: 12 human patients (n=12) with erythematotelangiectatic rosacea (n=10) or papulopustular rosacea (n=2). The patients consisted of 10 females (n=10) and 2 males (n=2). Patient age was as follows: mean (SD): 49.3 (12.3), (min, max): (31, 77), median: 50.0. All patients had skin type III as per Fitzpatrick classification (n=12). Study objectives: Primary objectives were to evaluate the efficacy and the cutaneous tolerability of the emulsion. Secondary objectives were to evaluate the usability of the emulsion and the cosmetic acceptance of the patients upon application of the emulsion. Efficacy assessments: Clinical evaluation of erythema, telangiectasia, count of inflammatory lesions (papules and pustules); stinging test; Transepidermal Water Loss (TEWL) measurement; photographs and skin imaging; and reflectance confocal microscopy (Demodex mites count). Safety assessments: Clinical assessments of safety (signs of irritation), subjective symptoms (stinging, burning and pruritus); questionnaire regarding usability and cosmetic acceptance; and record of related adverse effects at each post-baseline timepoint.

### Results

### Efficacy Results

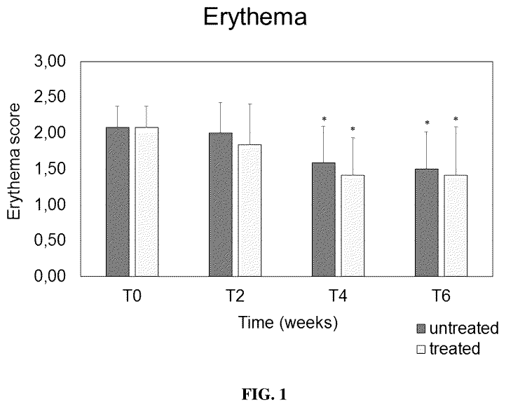

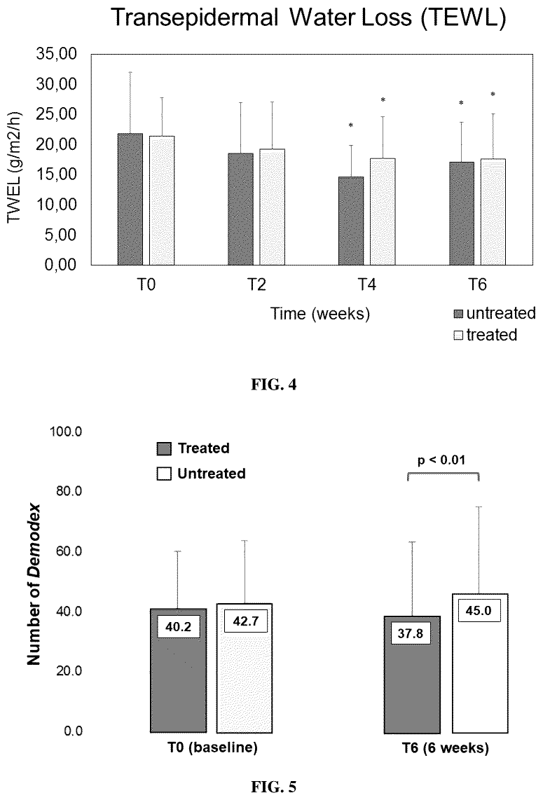

Erythema was significantly improved at four and six weeks of treatment by Em-A compared to baseline (*, p<0.001), as shown on FIG. 1. Transepidermal Water Loss (TEWL), which is a marker of the level of barrier properties of the skin (i.e., skin integrity), significantly decreased at four and six weeks compared to baseline (*, p<0.001), as shown on FIG. 4. Moreover, a significant decrease in the number of Demodex mites was observed at six weeks compared to baseline (p<0.01), as shown on FIG. 5.

Thus, these results clearly evidence that Em-A significantly reduce rosacea symptoms such as erythema, skin irritation, Demodex mite infestation and inflammation.

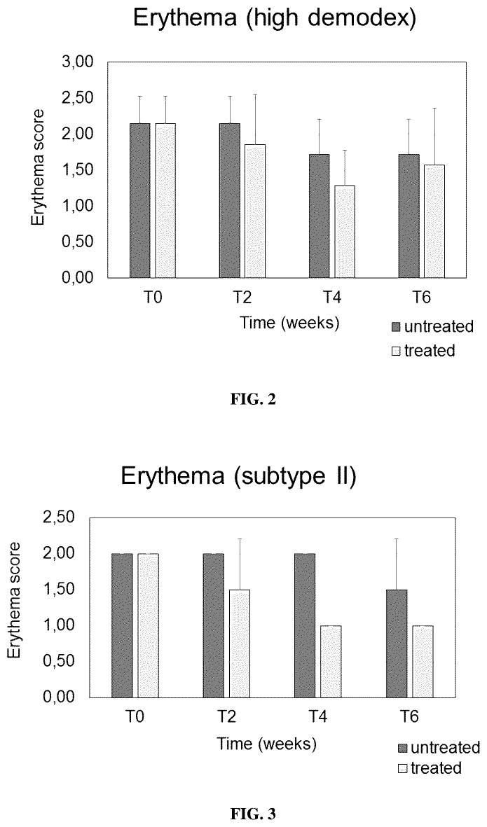

The study was originally designed with administration to only one side of the face of the patients in order to possibly use the other side as a control, but few significant differences between treated and untreated sides were observed in the end. In fact, it is relatively frequent to see improvements on both sides of the face while only one side is treated in the case of skin diseases: because rosacea has a neurovascular and immunological component, the affection can circulate between both halves of the faces and so does the therapeutic action. However, a tendency to higher improvement in the reduction of erythema on the treated side compared to untreated side has been identified for patients with a high count of demodex mites (at least 10 mites within a surface of 3×3 mm) and for patients having subtype II rosacea (with inflammatory lesions), as shown on FIG. 2 and FIG. 3 respectively.

### Safety Results

Treatment by Em-A did not reveal any local or general safety issues. Among the patients initially suffering from stings, lower stinging intensities were observed after six weeks of treatment by Em-A, compared to baseline. Moreover, according to the questionnaire results the patients were very satisfied about the usability of Em-A cream and the cosmetic acceptance was also very good. Record of adverse effects did not include any significant adverse effect of Em-A administration.

Thus, these results confirm the tolerance and safety of Em-A as shown by preliminary tests (Examples 1-3).

Therefore, this clinical study clearly evidences the efficacy of the emulsion according to the invention for use in the treatment of skin diseases such as rosacea, as well as excellent safety and patient acceptance thereof.

These results also confirm that no therapeutic agent is required in order to treat rosacea, when using the emulsion according to the invention.

1-15. (canceled)

16. A method for the treatment and/or prevention of a skin disease in a subject in need thereof, comprising a step of administering to said subject a therapeutically effective amount of an emulsion;
wherein said emulsion comprises at least one oil, at least one phospholipid, and at least one polymeric surfactant;
wherein said emulsion is an oil-in-water emulsion;
wherein said phospholipid does not comprise phosphatidylcholine (PC); and
wherein said emulsion does not comprise vitamin D3, or a derivative or precursor thereof.

17. The method according to claim 16, wherein said oil is selected from triglycerides, mineral oils, fatty acids, and mixtures thereof.

18. The method according to claim 16, wherein said oil comprises a mixture of triglycerides and fatty acids.

19. The method according to claim 16, wherein said phospholipid is selected from the group consisting of: dihexanoyl phosphatidylglycerol, dioctanoyl phosphatidylglycerol, didecanoyl phosphatidylglycerol, dilauroyl phosphatidylglycerol, lauroylmyristoyl phosphatidylglycerol, dimyristoyl phosphatidylglycerol, dipalmitoyl phosphatidylglycerol (DPPG), myristoylpalmitoyl phosphatidylglycerol, palmitoylstearoyl phosphatidylglycerol, myristoylstearoyl phosphatidylglycerol, distearoyl phosphatidylglycerol, dioleoyl phosphatidylglycerol, stearoyloleoyl-phosphatidylglycerol, palmitoyloleoyl phosphatidylglycerol, dilinoleoyl phosphatidylglycerol, dilinolenoyl phosphatidylglycerol, diarachidonoyl phosphatidylglycerol, didocosahexaenoyl phosphatidylglycerol, a fatty acid ester of phosphatidylglycerol, and a mixture thereof.

20. The method according to claim 16, wherein said phospholipid is dipalmitoyl phosphatidylglycerol (DPPG).

21. The method according to claim 16, wherein said polymeric surfactant is selected from the group consisting of an alkyl aryl polyether alcohol, a block copolymer of ethylene oxide and propylene oxide, a polyvinyl alcohol, a polyoxyethylene fatty acid ester, and a mixture thereof.

22. The method according to claim 16, wherein said polymeric surfactant is tyloxapol.

23. The method according to claim 16, wherein said emulsion comprises:
at least oils being medium chain triglycerides (MCT), stearic acid, oleic acid, linoleic acid, and alpha linolenic acid;
at least one phospholipid being dipalmitoyl phosphatidylglycerol (DPPG); and
at least one polymeric surfactant being tyloxapol.

24. The method according to claim 16, wherein said skin disease is rosacea.

25. The method according to claim 24, wherein said skin disease is papulopustular rosacea.

26. The method according to claim 16, wherein said method comprises a step of topical administration of said emulsion onto the skin of said subject.

27. An emulsion comprising
at least one oil comprising a mixture of triglycerides and fatty acids,
at least one phospholipid, and
at least one polymeric surfactant selected from an alkyl aryl polyether alcohol, a block copolymer of ethylene oxide and propylene oxide, a polyvinyl alcohol, a polyoxyethylene fatty acid ester and a mixture thereof;
wherein said emulsion is an oil-in-water emulsion;
wherein said phospholipid does not comprise phosphatidylcholine (PC); and
wherein said emulsion does not comprise vitamin D3, or a derivative or precursor thereof.

28. The emulsion according to claim 27, wherein said oil is a mixture of medium chain triglycerides (MCT), stearic acid, oleic acid, linoleic acid, and alpha linolenic acid.

29. The emulsion according to claim 27, wherein said phospholipid is dipalmitoyl phosphatidylglycerol (DPPG).

30. The emulsion according to claim 27, wherein said polymeric surfactant is tyloxapol.

31. The emulsion according to claim 27, wherein said emulsion comprises:
at least oils being medium chain triglycerides (MCT), stearic acid, oleic acid, linoleic acid, and alpha linolenic acid;
at least one phospholipid being dipalmitoyl phosphatidylglycerol (DPPG); and
at least one polymeric surfactant being tyloxapol.

32. The emulsion according to claim 27, wherein said emulsion comprises:
at least oils being medium chain triglycerides (MCT), linseed oil, corn oil, cetearyl alcohol, lanolin, and squalane;
at least one phospholipid being dipalmitoyl phosphatidylglycerol (DPPG);
at least polymeric surfactants being tyloxapol and polyethylene glycol 20 stearate;
at least one viscosifier being trehalose;
at least one pH adjusting agent being ascorbic acid; and
water.

33. The emulsion according to claim 27, wherein said emulsion does not comprise any therapeutic agent against a skin disease.

34. The emulsion according to claim 27, wherein said emulsion comprises a therapeutic agent against a skin disease.

35. The emulsion according to claim 34, wherein said emulsion comprises a therapeutic agent selected from the group consisting of: ivermectin, brimonidine, metronidazole, doxycycline, azelaic acid, tea tree oil, and mixtures thereof.
